(19) United States
(12) Patent Application Publication
(10) Pub. No.: US 2010/0111896 A1
(43) Pub. Date: May 6, 2010

(54) Items Containing A Human Pheromone Component

(76) Inventors: Barry J. Marenberg, New Providence, NJ (US); Katey A. Lang, New York, NY (US); Philip Y. Braginsky, Forest Hills, NY (US)

Correspondence Address: Barry Marenberg, 76 Brookside Dr., New Providence, NJ, 07974, US

(21) Appl. No.: 12/684,435

(22) Filed: Jan. 8, 2010

Related U.S. Application Data
(63) Continuation-in-part of application No. 12/094,287, now abandoned.

(51) Int. Cl. A61K 8/63; A61Q 13/00

(52) U.S. Cl. 424/84

(57) ABSTRACT

Food items, cosmetics and household items having a human pheromone component. Also disclosed are body washes, body splashes and body lotions that contain an incorporated human pheromone component or ingredient and methods for producing these products.

### RELATED APPLICATIONS

The present application is a continuation-in-part (CIP) application of U.S. application Ser. No. 12/094,287 filed May 20, 2008, which is the national phase entry application of PCT International Application No. PCT/US06/04035, filed Nov. 21, 2006, and PCT International Application No. PCT/US07/008104, filed Oct. 31, 2008 and which claims priority to U.S. Provisional Patent Application Ser. No. 60/788,679.

### FIELD OF THE INVENTION

The present invention relates to food items, cosmetics and household items having a human pheromone component. More particularly, the present invention is directed to a body wash, typically usable as a cleansing product while showering, which contains an incorporated human pheromone component or ingredient.

### BACKGROUND

Pheromones are chemicals emitted by living organisms to send messages to individuals of the same species. The classes most widely explored are the sex pheromones produced by female moths which are used to attract conspecific males for mating. Bombykol, the sex pheromone of the silkmoth, was first synthesized in 1959.

Most pheromones consist of blends of two or more chemicals which need to be emitted at exactly the right proportions to be biologically active. The female effluvia or sex gland can contain additional compounds which are related to the pheromone components and whose biological function is often unclear. On the other hand, many attractants of male moths have been discovered simply by field screening. In several cases it could later be shown that the attractant found with this technique was identical to the natural pheromone produced by the female. In most others, the composition of the true pheromone is still unknown.

While it is known that pheromone systems exist in insects, an increasing number of studies have shown that pheromones play a role in many species, including humans. Pheromones in humans are believed to be produced by the apocrine glands. These glands become functional after reaching puberty, which could explain why most people develop an attraction for others at that time. Pheromones could also be the reason why a person can sense “chemistry”, or feel an instant attraction or dislike when first meeting someone.

Certain compounds believed to have pheromone properties affect a specific behavioral or physiological response in human subjects, e.g., a reduction of negative affect, mood, and character traits. In one particular example, nasal administration provides for contacting neurochemical receptors of a heretofore poorly understood neuroendocrine structure, commonly known as the vomeronasal organ (“VNO); also known as “Jacobson's organ”), with one or more steroid(s) or with compositions containing the steroid(s). This organ is accessed through the nostrils of most higher animals—from snakes to humans, and has been associated, inter alia, with pheromone reception in certain species (see generally Muller-Schwarze & Silverstein, Chemical Signals, Plenum Press, New York (1980)). The axons of the neuroepithelia of the vomeronasal organ, located supra palatinal, form the vomeronasal nerve and have direct synaptic connection to the accessory olfactory bulb and indirect input from there to the cortico-medial amygdaloid basal forebrain and hypothalamic nuclei of the brain. The distal axons of terminalis nerve neurons may also serve as neurochemical receptors in the VNO. Stensaas, L., et al., J. Steroid Biochem. and Molec. Biol. (1991) 39:553. This nerve has direct synaptic connection with the hypothalamus.

### SUMMARY OF THE INVENTION

The invention provides food items, such as candies, which are formulated with and/or coated with a human pheromone substance. The food items of the present invention will cause the person eating the food item to experience a unique, mood-altering effect which may include libido enhancement. This effect may also be an overall sense of well-being or may one of heightened sexual attractiveness.

The invention also provides cosmetics and household items which have an incorporated human pheromone component that elicits a desired positive feeling or sensation. More particularly, the present invention is directed to body washes, body splashes and body lotions that contain an incorporated human pheromone component or ingredient.

### DESCRIPTION OF THE INVENTION

As used herein, a “pheromone” is a substance that provides chemical means of communication between members of the same species through secretion and peripheral chemoreception. In mammals pheromones are usually detected by receptors in the vomeronasal organ of the nose. Commonly, pheromones effect development, reproduction and related behaviors. The pheromone compositions according to the present invention are intended to cause a change in human hypothalamic function, thereby altering certain behavior and physiology mediated by the hypothalamus of individuals.

In addition to physiological responses, pheromones can be identified by their species specific binding to receptors in the vomeronasal organ (VNO). Thus, human pheromones bind to human receptors. This can be demonstrated by measuring the change in the summated potential of neuroepithelial tissue in the presence of the pheromone. Human pheromones induce a change of at least about −5 millivolts in human neuroepithelial tissue of the appropriate sex (The binding of pheromones is generally sexually dimorphic, i.e., different in the effect of, or response to, a compound or composition between males and females of the same species). Naturally occurring human pheromones induce sexually dimorphic changes in receptor binding potential in vivo in the human VNO. Naturally occurring human pheromones can be extracted and purified from human skin and they can also be synthesized. “Human pheromones” are pheromones which are naturally occurring in humans and effective as a specifically binding ligand in human VNO tissue, regardless of how the pheromone was obtained. Thus, both a synthesized and purified molecule may be considered a human pheromone.

In one embodiment, chocolate candy is provided with a pheromone ingredient (or component) or which has a pheromone coating. The pheromone is colorless, tasteless and odorless and thus does not detract in any way from the appearance, smell or taste of the chocolates.

In another embodiment the pheromone composition will be incorporated into or coated upon candies such as lollipops and hard sucking candies, although the invention is not limited in this respect. The primary ingredient in these candies is sugar as sucrose. Turning the sugar into candy involves dissolving it in water, concentrating this solution through cooking, and subsequently allowing the mass either to form mutable solid or to recrystallize. Basic candy making is done in many known ways including, but not limited to, cooking a sugar solution to a specific temperature to form a supersaturated solution with a known solids content. When this solution cools the sugar's solubility decreases and the sugar crystallizes out of solution. Recrystallizing, also known as graining is controlled by varying how the supersaturated syrup is physically treated while it cools. Suspending a length of string into the solution and letting it slowly cool undisturbed will allow the sugar to recrystallize into large crystals on the string to form rock candy. Cooling that same solution quickly with agitation will cause fine crystals to form and be suspended in a saturated sugar syrup to become fondant. Fondant is used in confectionery creme centers. The present invention provides for the pheromone composition, component or ingredient to be incorporated into the sugar solution and recrystallized, or it may remain in the fondant. Alternatively the pheromone component or ingredient may be used to coat the recrystallized candy.

Candies can be grouped into four basic types. Ungrained candies are candies in which the sugar isn't crystallized. These include hard candies, as well as chewy candies such as toffee and caramel. Hard candies start with a basic sugar/glucose syrup blend cooked down to around 2% moisture and blended with flavors and colors. Varying the ratio of sweeteners produces different textures and different stability. Cooking the basic sugar/glucose syrup blend to a moisture level between 3% to 15% forms the basis of a chewy non-grained candy. Cooking to these higher moisture levels will not favor the production of invert sugar, as is the case with hard candies. As a result, the addition of glucose syrup is critical to prevent undesired graining in chewy candies. The key is basic sugar/glucose syrup ratio. The more sugar crystals, the tougher and grainier the candy is. The more glucose syrup it will be chewier. Caramels, on the other hand, require milk and fat in the formula to provide flavor and color. In caramels, the Maillard reaction that goes on with the reducing sugars and milk proteins is important for developing both flavor and color. The higher the amount of reducing sugar (higher DE) Maillard reaction will speed up.

Grained candies include products such as after-dinner mints, fondants/creme centers, and fudge. Grained candy formulas are similar to those of chewy candies. To promote crystallization, however, the formula must have a higher level of sugar solids and the process usually includes agitation.

Jelly candies, such as gumdrops, include starch, pectin or gelatin in the basic formula to achieve yet another unique candy texture through the selection of the stabilizer system. The type of gelling agent use will make the biggest difference. A gum-based jelly candy will be chewier, while a starch jelly candy will be shorter. It is also will be stickier. Pectin jellies make a high quality jelly candy with a good texture and good flavor release (unique texture which consists of a relatively soft bite with a short texture and rapid meltaway). Pectin is used more in higher priced, premium jelly candies and centers. A gelatin-based jelly candy gives a greater clarity. The gelatin normally used has a medium to high bloom or gel strength. A wide range of textures can be generated using either lower concentrations of a high-bloom gelatin, which gives a more tender and short texture jelly candy or higher concentrations of a low-bloom which gives a more elastic and chewy texture jelly candy.

Aerated candies comprise a large group of products. Aerated candies can be based on a syrup mixture that is either ungrained or grained. These candies are unified by the fact that they all require the inclusion of air into the basic syrup matrix during processing. Aeration itself provides a shorter texture, modifies mouthfeel and reduces stickiness of the candy. Aeration is normally in two means: chemical or mechanical. In chemical aeration, the candy will contain ingredients such as sodium bicarbonate to leaven it like a baked product. In mechanical aeration, the formula will contain a foaming agent and/or a stabilizer, and it will have air incorporated using a continuous pressure beater. Aerated candies may be either grained or ungrained, their sweetener blends can be quite different from one another. Marshmallow provides a good example because it is made in both forms. A traditional marshmallow might contain about 60% glucose syrup, 30% sugar, and 1% to 2% gelatin. The glucose syrup/sugar ratio here will provide only about 35% to 40% solids in order to prevent crystallization. Crystallization can be further avoided with selection of a higher conversion glucose syrup which contribute more invert sugar to the formula. A grained marshmallow, you simply increase the sugar ratio to the point where it will crystallize about 60% to 65%. Whipping it and seed it with a little powdered sugar. As it cools, the sugar crystallizes out to form the grained marshmallow. Aerating agents, which often are protein-based ingredients such as egg albumen and soy protein are used. Stabilizers are often used in aerated candies. Gelatin, is a key stabilizer because gelatin in this application decreases surface tension and it gives the cell walls resistance to deformation and maintains the structure. Water-binding capabilities also help to increase shelf life.

The present invention contemplates adding a pheromone component to all of these types of candy, either by incorporation or coating.

The pheromone composition may also be incorporated into coffee and tea to give off a sensuous odor when the coffee or tea is removed from their respective package, and when they are heated for consumption. Coffee is processed from beans grown in various regions of the world including Africa and South America. After the beans are ripe and picked from the plant they are typically processed and then roasted for consumption. In this embodiment, the pheromone component may be added during the processing and/or roasting process. It is also contemplated that beans may be grown which incorporate the pheromone component.

Tea is a beverage made by steeping processed leaves, buds or twigs of the tea bush Camellia sinensis in hot water for a few minutes. Although tea originated in China and India, it is now grown in many places throughout the world. The processing of tea can include oxidation (fermentation), heating, drying and the addition of other herbs, flowers, spices and fruits. There are four types of true tea: black tea, oolong tea, green tea, and white tea. The term herbal tea usually refers to infusions of fruit or herbs such as rosehip tea, chamomile tea and Jiaogulan that contain no tea leaves. In this embodiment, the pheromone component may be added during the processing process. It is also contemplated that tea may be grown which incorporates the pheromone component.

The present invention also contemplates flowers or plants which may be grown and cultivated with a human pheromone composition which emanates from the flower or plant to fill a room with the pheromone. In another embodiment, a powder of spray formulation comprising a human pheromone composition can be used to sprinkle or spray on existing flowers or plants.

Candles may also be made having a pheromone component that emanates from the candle when the candle is lit. Candles can be made in a variety of ways and are typically formed from waxes and/or oils or a combination of the two. The present invention is not limited in the methodology of making the candle. The pheromone component is simply added as an ingredient to any candle-making method known in the art.

Soaps, body washes, lotions and creams may also be formulated with the one or more pheromones in accordance with the present invention. This embodiment of the invention is directed to the incorporation of a pheromone component into these cleansing products and is not limited by the method of making or manufacturing the product.

Soap is a surfactant used in conjunction with water for washing and cleaning It usually comes in a gas moulded form, termed bars due to its historic and most typical shape. The use of thick liquid soap has also become widespread, especially from soap dispensers in public washrooms. Applied to a soiled surface, soapy water effectively holds particles in suspension so the whole of it can be rinsed off with clean water. In the developed world, synthetic detergents have superseded soap as a laundry aid.

Soap is derived from either oils or fats. Sodium tallowate, a common ingredient in many soaps, is in fact derived from rendered beef fat. Soap can also be made of vegetable oils, such as olive oil. Soap made entirely from such oils, or nearly so, is called castile soap. Many soaps are mixtures of sodium (soda) or potassium (potash) salts of fatty acids which can be derived from oils or fats by reacting them with an alkali (such as sodium or potassium hydroxide) at 80°-100° C. in a process known as saponification. The fats are hydrolyzed by the base, yielding glycerol and crude soap. Historically, the alkali used was potassium made from the deliberate burning of vegetation such as bracken, or from wood ashes.

In another embodiment, a body wash composition is provided and which may contain any of a variety of ingredients to cleanse skin and impart desired conditioning properties to the skin. The ingredients used in a personal cleansing product, such body wash and hand soap, can typically be divided into four major categories: Primary surfactants, secondary surfactants, specialty ingredients and minor ingredients.

Primary surfactants perform two important functions—cleansing and foaming. Desirable body wash properties may also include quick and easy miscibility with water; instant flash foaming; nice, creamy lather that feels silky soft upon application, effective cleaning of soils from the hair and/or skin, quick and easy rinseability, and soft and silky afterfeel.

Alkyl sulfates and alkyl ether sulfates such as sodium/ammonium lauryl sulfates and sodium/ammonium laureth sulfates, are cost-effective primary surfactants which are used in many body wash products. Alkyl sulfates and alkyl ether sulfates meet consumer demand for foam and lather as well as cleansing. These reasonably priced, high foaming surfactants have excellent cleansing properties. The shampoo or body wash composition also contains at least one anionic surfactant. Any anionic surfactant may be used that is dermatologically compatible with skin or hair. Sodium lauryl sulfate, sodium laureth sulfate, ammonium lauryl sulfate, ammonium laureth sulfate are commonly known anionic surfactants.

By “dermatologically compatible” it is meant that the compound does not cause irritation when applied to the skin for the purpose of cleaning and then is subsequently removed within about 30 minutes of application. The anionic surfactant is preferably present at a level of from about 1 weight percent to about 40 weight percent, more preferably from about 5 weight percent to about 30 weight percent, and most preferably from about 5 weight percent to about weight percent, based on the total weight of the composition.

Many formulators prefer to use a combination of lauryl sulfate and lauryl ether sulfate. Lauryl sulfates produce creamier foam with higher volumes of small bubbles compared to ether sulfates. Conversely, ether sulfates, particularly the two and three moles (EO), produce better flash foam. To develop a product with optimum yet balanced lather profile, lauryl and lauryl ether sulfates are added in a certain range of ratios.

In one example, the body wash in accordance with the present invention will contain an anionic cleansing surfactant, such as an alkyl ether sulfate or an alkyl sulfate, such as sodium lauryl ether sulfate or sodium lauryl sulfate; a polymeric cationic conditioning compound, such as a quaternized guar gum; and a quaternized phosphate ester in an aqueous carrier is disclosed. The composition is used in a method to cleanse the skin and to impart conditioning properties to the skin. More particularly, in one embodiment the body wash of the present invention is directed to a body wash composition comprising: (a) an anionic surfactant, like an alkyl ether sulfate, such as sodium lauryl ether sulfate; (b) a polymeric cationic conditioning compound, such as a quaternized guar gum; and (c) a quaternized phosphate ester, in an aqueous carrier. The body wash compositions of the present invention exhibit a minimal interaction, if any, between the cationic components and the anionic components, making the anionic components and the cationic components available to effectively cleanse and condition the skin.

For sodium lauryl sulfate and sodium laureth-2 sulfate a good starting ratio is 3:7 on an active surfactant amount basis. This combination of lauryl and laureth sulfate provides a nice, well-balanced lather profile. This ratio is also a good starting point for viscosity response and short flow (non stringy) properties of the final product. Ammonium lauryl/laureth sulfates may be slightly better than their sodium counterparts in amount of foam generated, clarity at lower temperatures and viscosity response in certain formulas but they can release ammonia at higher pH. The present invention has been formulated using sodium laureth sulfate but the invention is not limited to this specific primary surfactant and other primary/anionic surfactants may be used without detracting from the invention.

Secondary surfactants typically impart increased viscosity, stabilized foam and reduced skin and eye irritation to a formulation. Personal cleansing systems based on alkyl sulfates and alkyl ether sulfates are relatively easy and cost effective for viscosity buildup. Generally speaking, a 10% active sulfate system will require about 1-2% of alkanolamide and 1-3% sodium chloride to reach or exceed a viscosity of 10,000 cps. Regarding rheology, this 3:7 lauryl to laureth sulfate ratio imparts a nice short flow for the system. Higher amounts of lauryl sulfates may cause stringy flow whereas the higher amounts of laureth sulfate will not have optimized creaminess of the lather and may not build maximum viscosity.

Salts, such as sodium chloride, increase the viscosity of sulfate-amide systems. It is always good to run a salt viscosity curve for each formula, which can be done by adding the salt to the formula in increments of 0.2% or so. After a certain amount of salt has been added, the viscosity peaks and any more salt will actually reduce the viscosity rather than increase it. This type of curve is useful for adjusting the viscosity of the product. Some formulators purposely exceed the curve in order to reduce the stringy character of the formula. Stringy flow can also be corrected by adding a little polysorbate 20 or propylene glycol to the formula. However, these materials will reduce the viscosity as well, which is why they are added in very low quantities such as 0.1-0.5%.

Betaines, like cocamidopropyl betaine (CAPB), are also good secondary surfactants because of their high-foaming, viscosity-building and mildness-providing properties. In some formulas, CAPB acts as sole viscosity builder. So, for example, “amide free” formulas can be made with CAPB. When used in conjunction with alkanolamides, CAPB increases the viscosity more than can be achieved with the amide alone. As a general rule, 10-20% of alkanolamide and 10-20% of active CAPB based on the active amounts of the primary surfactants is a good starting point for formulation development. For example, with 10% total actives of alkyl/alkyl ether sulfates, 1-2% of an alkanolamide and 1-2% active CAPB (or about 3-6% as supplied) is needed.

Secondary surfactants also typically improve the mildness properties of bath and shower products. Amphoteric surfactants, such as amphoacetates and amphopropionates, are used for mildness. Sulfosuccinates, alkyl polyglucosides, betaines, sultaines, sarcosinates, isethionates, taurates, ethoxylated sorbitan esters and amino acid-based surfactants improve mildness. An important aspect of a personal cleansing formula is a good lather profile. That is why it is important to keep some of alkyl/alkyl ether sulfate in the formula and incorporate one or a combination of some mild surfactants. It is contemplated that in formulating a body wash according to the present invention, one or more and/or a mixture of surfactants may be utilized. One particularly preferred surfactant is PPG-2 hydroxyethyl coco/isostearamide, but the invention is not limited in this respect and other secondary surfactants may be used without detracting from the inventive formulation.

The third and fourth categories of ingredients in body washes and shower products, specialty ingredients and minor ingredients include a wide range of materials such as conditioning agents, special effect additives, proteins and vitamins. Body washes may also typically use conditioning agents such as fatty amines, fatty quats, polydimethyl siloxanes, organo modified siloxanes (amino functional, quaternized silicones) and quaternized polymers (cellulose and guar). Body washes using quats and amines must be compatible with any anionic surfactants in the formula. A formulator must carefully choose the quality and quantity of fatty quat and fatty amine to avoid heavy buildup, dulling, and flattening effects in the final product. The addition of quaternized polymers, such as quaternized cellulose, provide a light conditioning effect to body and head hair and impart static control and easy combing if the body wash is also used as a shampoo.

Examples of synthetic quaternized polymers include, but are not limited to, polyquaternium-1, polyquaternium-2, polyquaternium-5, polyquaternium-6, polyquaternium-7, polyquaternium-8, polyquaternium-9, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-27, polyquaternium-28, polyquaternium-29, polyquaternium-30, and mixtures thereof, wherein the compound designation is the name adopted for the compound by the Cosmetic, Toiletry and Fragrance Association, and found in the CTFA International Cosmetic Ingredient Dictionary, J. Nikitakis, ed., Cosmetic, Toiletry and Fragrance Association, Inc., Washington, D.C. (1991).

In some embodiments of the body wash according to the present invention , the body wash composition contains an amphoteric surfactant, zwitterionic surfactant, or combinations thereof. Preferably, the total amount of amphoteric and zwitterionic surfactant is from 0 to about 5 weight percent and more preferably from about 1 to about 4, based on the total weight of the composition. Examples of suitable amphoteric or zwitterionic surfactants for use in the present invention include alkyl betaines, alkylamidobetaines, aminopropionates, aminoglycinates, imidazolinium betaines, sulfobetaines or combinations thereof. One or more nonionic surfactants may also be added to the body wash composition.

Examples of amphoteric or zwitterionic surfactants useful in the present invention include sodium 3-dodecyl-aminopropionate, sodium 3-dodecylaminopropane sulfonate, sodium lauroamphoacetate, coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl)sulfopropyl betaine or combinations thereof. Most preferably, the zwitterionic or amphoteric surfactant is cocoamidopropyl betaine, oleamidopropyl betaine, or combinations thereof.

Body wash formulations may also include special additives and/or minor ingredients. Some of these materials include:

Proteins: Hydrolyzed vegetable protein, hydrolyzed wheat protein, hydrolyzed milk protein, hydrolyzed silk and hydrolyzed collagen.

Vitamins: Panthenol, biotin, vitamin E acetate, vitamin A and D palmitate.

Moisturizers/humectants: glycerin, propylene glycol, sodium PCA, amino acid based surfactants and hylauronic acid.

Emollients: Esters like isopropyl myristate, decyl oleate and C12-15 alkyl benzoate.

Natural oils and botanicals: Jojoba oil, aloe vera oil, safflower oil, almond oil, chamomile, rosemary and hops.

Aromatherapy: Lavender, ylang ylang, patchouli and other essential oils.

Minor ingredients include preservatives, perfume, dye, pH adjusters and chelating agents.

For example, additional ingredients in the body wash formulation of the present invention include glycerin, PEG-8, sodium chloride, citric acid, DMDM hydantoin and tetrasodium EDTA. glyercerin, also commonly referred to as glycerol or glycerine is an organic compound. It is a colorless, odorless, viscous liquid that is widely used in pharmaceutical formulations. Glycerine has three hydrophilic hydroxyl groups that are responsible for its solubility in water and its hygroscopic nature.

PEG-8, or propylene glycol cocoate is a chemical compound produced by the esterification of polyalkyl alcohols with lauric acid and a commonly used emulsifying agent. PEG-8 is well known for its use in cosmetics to form emulsions, which it does by reducing surface tension in the substances of which an emulsified form is required.

Citric acid is a weak organic acid that is typically used in shampoos and body washes as an environmentally benign cleaning agent. Tetrasodium EDTA is commonly used in shampoos and body washes as a penetrating agent.

DMDM hydantoin is an antimicrobial formaldehyde releaser preservative with the trade name Glydant. DMDM hydantoin is an organic compound that belongs to a class of compounds known as hydantoins. It is used in the cosmetics industry and found in products like shampoos, hair conditioners and skin care products to prevent molds, mildews, and bacterial spoilage.

Numerous fragrances are added to the formulation to provide a pleasant and attractive scent to the body wash. Additionally, one or more pigments and coloring agents are also added such as, but not limited to, Red 40, Red 33 and Blue 1.

The body wash of the present invention may also include plant extracts; vitamins such as Vitamins A, B, C, or E, or combinations thereof; preservatives such methylchloroisothiazolinone, methylisothiazolinone, or diazolidinyl urea or combinations thereof; humectants, such as sorbitol, glycerin, propylene glycol, or butylene glycol or combinations thereof; light stabilizers; dyes; pearlescers, such as ethylene glycol monostearate or distearate; antioxidants; thickeners such as polysaccharides, including for example, xanthan gum, guar gum, agar gum, alginates, tyloses, or celluloses such as carboxymethyl cellulose, or hydroxyethyl cellulose; polyethylene glycol monoesters or diesters of fatty acids.

In another embodiment, a human pheromone composition in accordance with the present invention may be incorporated into lipstick, lip gloss, lip balm or lip plumper, which is applied on a wearer's lips.

The primary ingredients found in lipstick are wax, oil, alcohol, and pigment. The wax used usually involves some combination of three types—beeswax, candelilla wax, or the more expensive carnauba. Wax enables the mixture to be formed into the easily recognized shape of the cosmetic. Oils such as mineral, caster, lanolin, or vegetable are added to the wax. Fragrance and pigment are also added, as are preservatives and antioxidants, which prevent lipstick from becoming rancid. And while every lipstick contains these components, a wide variety of other ingredients can also be included to make the substance smoother or glossy or to moisten the lips. In accordance with this embodiment, a human pheromone component is added as an ingredient during the production of the lipstick

Another embodiment contemplates a pheromone spray or powder which may be applied to a condom to enhance libido and enhance sexual intercourse.

The powder or spray composition comprising one or more human pheromones may be formed using synthetic methods known to those of ordinary skill in the art. Although not intending to be limited in any way, the powder or spray composition in accordance with the present invention may be formed via extraction, filtration, crystallization and other synthetic methods. In addition to being applied to the items described above, the powder or spray could be sprinkled/sprayed around a bedroom and on garments such as lingerie, intimate apparel and sleepwear in order to provide a sensuous atmosphere.

The present invention also contemplates the application of human pheromone ingredient, component and compositions to a substrate and article, such as an advertisement card in a magazine. In this embodiment, one or more pheromone compositions are deposited on a substrate. A film forming agent may serve to microencapsulate the composition and bond it to the substrate. When the film forming agent dries, it protects the pheromone composition. A cover sheet may then cover the film forming agent. Access to the pheromone composition can be provided by a tear strip. When the strip is torn off or separated from the film forming agent, the pheromone composition is released. Application of the pheromone on a substrate allows it to be mass distributed through the mail and as magazine inserts and the like. Such use will provide a means for mass distribution of the pheromone compounds.

The term “pheromone” as used herein refers to a natural or synthetic compound having a steroid scaffold (cyclopentanoperhydrophenanthrene ring system) with a plurality of substituents and optional double bonds, wherein the compound elicits in vivo a measurable response in the vomeronasal organ (e.g., measured as negative receptor binding potential as described in U.S. Pat. No. 5,272,134, incorporated by reference herein) of an animal having a vomeronasal organ. The term “human pheromone” as also used herein refers to a steroid pheromone that elicits an in vivo measurable response in the human vomeronasal organ.

The present invention is not limited in the pheromone composition used. Exemplary pheromone compounds include, but are not limited to, estrene steroids, such as those described in U.S. Pat. No. 5,925,774, which is incorporated by reference herein; vomeropherins, such as 19-nor-cholane steroids as described in U.S. Pat. Nos. 6,437,156 and 6,352,980, which are incorporated herein by reference in their entirety; and androstene and androstenedione compounds such as those described in U.S. Pat. Nos. 4,071,624 and 5,272,134, which are also incorporated by reference herein in their entirety, as well as copulins, which include but are not limited to, short chain fatty acids. Additionally, as described in U.S. Pat. No. 6,242,619, which is incorporated by reference in its entirety, several 19-nor-pregnanes have also demonstrated the capacity to neurochemically alter the hypothalamic function in an individual.

A non-exhaustive list of androstene and androstene dione steroids which may be utilized in accordance with the present invention include:
17β-hydroxy-1-methyl-androst-4-ene-3,19-dione,
17α-ethyl-17β-hydroxy-4-methyl-androst-4-ene-3,19-dione,
1,7-dimethyl-androst-4-ene-3,17,19-trione,
17β-hydroxy-6-methyl-17α-(1′-propenyl)-androst-4-ene-3,19-dione 17-acetate,
17α-hexyl-1,4-dimethyl-androst-4-ene-3,19-dione, 17α-ethynyl-17β-hydroxy-7-methyl-androst-4-ene-3,19-dione,
1,4,6-trimethyl-androst-4-ene-3,17,19-trione, 1,7-dimethyl-17β-(4′-tetrahydropyranyloxy)androst-4-ene-3,19-dione,
17β-hydroxy-1,4,6,7-tetramethyl-androst-4-ene-3,19-dione 17-decanoate, 17α-butyl-17β-(1′-cycloheptenyloxy)-androst-4-ene-3,19-dione,
1,7-dimethyl-17α-(1′-propenyl)-17β-triphenylsilyloxy-androst-4-ene-3,19-dione,
17β-hydroxy-7,17α-dimethyl-androst-4-ene-3,19-dione,
17β-(1′-ethoxycyclopentyloxy)-6-methyl-androst-4-ene-3,19-dione,
17α-ethynyl-1-methyl-17β-trimethylsiloxy-androst-4-ene-3,19-dione, and
1,6,7-trimethyl-androst-4-ene-3,17,19-trione.

A non-limiting list of vomeropherins which may be used in accordance with the present invention include:
19-norchola-4,20-dien-3-one,
19-norchola-1,3,5(10),20-tetraen-3-ol,
19-norchola-4,20-dien-3β-ol,
19-norchola-1,3,5(10),6,20-pentaen-3-ol,
19-norchola-4,9,20-trien-3-one,
19-nor-3-hydroxychola-1,3,5(10),20-tetraen-6-one,
19-nor-10β-hydroxychola-4,20dien-3-one,
19-nor-3-methoxychola-2,5(10),20-triene,
19-norchola-1,3,5(10),7,20-pentaen-3-ol,
19-norchola-1,3,5,7,9,20-hexaen-3-ol,
19-norchola-5(10),20-dien-3β-ol,
19-norchola-1,3,5(10),20-tetraen-3,6β-diol,
19-norchola-5(10),20-dien-3-one,
19-norchola-1,3,5(10),9(11),20-pentaen-3-ol,
19,21-bisnorchola-4,17(20)-dien-3-one,
19,21-bisnorchola-4,17(20)-dien-3β-ol,
19,21-bisnorchola-1,3,5(10),6,17(20)-pentaen-3-ol,
19,21-bisnorchola-4,9,17(20)-trien-3-one,
19,21-bisnor-3-hydroxychola-1,3,5(10),17(20)-tetraen-6-one,
19,21-bisnor-10β-hydroxychola-4,17(20)-dien-3-one,
19,21-bisnor-3-methoxychola-2,5(10),17(20)-triene,
19,21-bisnorchola-1,3,5(10),7,17(20)-pentaen-3-ol,
19,21-bisnorchola-1,3,5,7,9,17(20)-hexaen-3-ol,
19,21-bisnorchola-5(10),17(20)-dien-3β-ol,
19,21-bisnorchola-1,3,5(10),17(20)-tetraen-3,6β-diol,
19,21-bisnorchola-5(10),17(20)-dien-3-one,
19,21-bisnorchola-1,3,5(10),9(11),17(20)-pentaen-3-ol,
19,21-bisnorchol-4-en-3-one,
19,21-bisnorchol-4-en-3β-ol, 19,21-bisnorchola-1,3,5(10),6-tetraen-3-ol,
19,21-bisnorchola-4,9-dien-3-one, 19,21-bisnor-3-hydroxychola-1,3,5(10)-trien-6-one,
19,21-bisnor-10β-hydroxychol-4-en-3-one,
19,21-bisnor-3-methoxychola-2,5-(10)-diene,
19,21-bisnorchola-1,3,5(10),7-tetraen-3-ol,
19,21-bisnorchola-1,3,5,7,9-pentaen-3-ol,
19,21-bisnorchol-5(10)-en-3β-ol,
19,21-bisnorchola-1,3,5(10)-trien-3,6β-diol,
19,21-bisnorchol-5(10)-en-3-one,
19,21-bisnorchola-1,3,5(10),9(11)-tetraen-3-ol,
19,21-bisnorchola-4,16-dien-3-one,
19,21-bisnorchola-1,3,5(10),16-tetraen-3-ol,
19,21-bisnorchola-4,16-dien-3β-ol,
19,21-bisnorchola-1,3,5(10),6,16-pentaen-3-ol,
19,21-bisnorchola-4,9,16-trien-3-one,
19,21-bisnor-3-hydroxychola-1,3,5(10),16-tetraen-6-one,
19,21-bisnor-10β-hydrocxychola-4,16-dien-3-one,
19,21-bisnor-3-methoxychola-2,5(10),16-triene,
19,21-bisnorchola-1,3,5(10),7,16-pentaen-3-ol,
19,21-bisnorchola-1,3,5,7,9,16-hexaen-3-ol,
19,21-bisnorchola-5(10), 16-dien-3β-ol,
19,21-bisnorchola-1,3,5(10),16-tetraen-3,6β-diol,
19,21-bisnorchola-5(10),16-dien-3-one,
19,21-bisnorchola-1,3,5(10),9(11),16-pentaen-3-ol,
19,21-bisnorchola-4,16-dien-20(22)-yn-3-one,
19,21-bisnorchola-1,3,5(10),16-tetraen-20(22)-yn-3-ol,
19,21-bisnorchola-4,16-dien-20(22)-yn-3β-ol,
19,21-bisnorchola-1,3,5(10),6,16-pentaen-20(22)-yn-3-ol,
19,21-bisnorchola-4,9,16-trien-20(22)-yn-3-one,
19,21-bisnor-3-hydroxychola-1,3,5(10),16-tetraen-20(22)-yn-6-one,
9,21-bisnor-10β-hydroxychola-4,16-dien-20(22)-yn-3-one,
19,21-bisnor-3-methoxychola-2,5(10),16-trien-20(22)-yne,
19,21-bisnorchola-1,3,5(10),7,16-pentaen-20(22)-yn-3-ol,
19,21-bisnorchola-1,3,5,7,9,16-hexaen-20(22)-yn-3-ol,
19,21-bisnorchola-5(10),16-dien-20(22)-yn-3-ol,
19,21-bisnorchola-1,3,5(10),16-tetraen-20(22)-yn-3,6β-diol,
19,21-bisnorchola-5(10),16-dien-20(22)-yn-3-one,
19,21-bisnorchola-1,3,5(10),9(11), 6-pentaen-20(22)-yn-3-ol, and
19,21-bisnor-3-methoxychola-1,3,5(10),16-tetraene.

A non-limiting list of estrenes for use in accordance with the present invention may include:
estra-1,3,5(10),6,16-pentaen-3-yl acetate,
estra-1,3,5(10),7-tetraen-3-ol,
3-hydroxy estra-1,3,5(10),16-tetraen-6-one,
6-oxo estra-1,3,5(10),16-tetraen-3-yl acetate,
17-methylene estra-1,3,5,7,9-pentaen-3-ol,
estra-1,3,5(10),16-tetraen-3,6b-diol,
6b-hydroxyestra-1,3,5(10),16-tetraen-3-yl acetate,
estra-4,16-dien-3b-ol,
17-methylene-6-oxoestra-1,3,5(10)-trien-3-yl acetate,
estra-4,9,16-trien-3-one,
estra-1,3,5,7,9,16-hexaen-3-ol,
estra-1,3,5(10),6-tetraen-3-ol,
3-methoxyestra-2,5(10),16-triene,
10-hydroxyestra-4,16-dien-3-one,
17-methyleneestra-1,3,5(10),7-tetraen-3-ol,
estra-5(10),16-dien-3 a-ol,
estra-5(10),16-dien-3 a-ol,
17-methyleneestra-4-en-3-one,
estra-1,3,5,7,9,16-hexaen-3-yl acetate,
17-methylgona-4,13(17)-dien-3b-ol,
estra-1,3,5,(10),7,16-pentaen-3-ol,
3-methoxy-17-methyleneestra-2,5(10)-diene,
17-methyleneestra-4-en-3b-ol,
17-methyleneestra-1,3,5(10)-triene-3,6b-diol,
estra-1,3,5(10),7,16-pentaen-3-yl acetate,
estra-1,3,5(10),6,16-pentaen-3-ol, and
17-methyleneestra-1,3,5(10),7-tetraen-3-yl acetate

A non-limiting list of 19-nor-pregnanes which may be used in accordance with the present invention include:
19-norpregna-4,20-dien-3-ol,
19-norpregna-4,17(20)-dien-3-ol,
19-norpregna-4,16-dien-3-ol,
19-norpregn-4-en-3-ol,
19-norpregn-4-en-20-yn-3-ol,
19-norpregna-4,16,20-trien-3-ol,
19-norpregna-1,3,5(10),6,20-pentaen-3-ol,
19-norpregna-1,3,5(10),6,17(20)-pentaen-3-ol,
19-norpregna-1,3,5(10),6,16-pentaen-3-ol,
19-norpregna-1,3,5(10),6-tetraen-3-ol,
19-norpregna-1,3,5(10),6-tetraen-20-yn-3-ol,
19-norpregna-1,3,5(10),6,16,20-hexaen-3-ol,
19-norpregna-1,3,5(10),6,17(20),20 hexaen-3 ol,
19-norpregna-1,3,5(10),6,16-pentaen-20-yn-3-ol,
19-norpregna-4,9,20-trien-3-one,
19-norpregna-4,9,17(20)-trien-3-one,
19-norpregna-4,9,16-trien-3-one,
19-norpregna-4,9-dien-3-one,
19-norpregna-4,9-dien-20-yn-3-one,
19-norpregna-4,9,16,20-tetraen-3-one,
19-norpregna-4,9,17(20),20-tetraen-3-one,
19-norpregna-4,9,16-trien-20-yn-3-one,
3-hydroxy-19-norpregna-1,3,5(10),20-tetraen-6-one,
3-hydroxy-19-norpregna-1,3,5(10),17(20)-tetraen-6-one,3-hydroxy-19-norpregna-1,3,5(10),16-tetraen-6-one,
3hydroxy-19norpregna-1,3,5(10)-trien-6-one,
3-hydroxy-19-norpregna-1,3,5(10)-trien-20-yn-6-one,
3-hydroxy-19-norpregna-1,3,5(10),16,20-pentaen-6-one,
3-hydroxy-19-norpregna-1,3,5(10),17(20),20-pentaen-6-one,
3-hydroxy-19-norpregna-1,3,5(10),16-tetraen-20-yn-6-one,
10-hydroxy-19-norpregna-4,20-dien-3-one,
10-hydroxy-19-norpregna-4,17(20)-dien-3-one,
10-hydroxy-19-norpregna-4,16-dien-3-one,
10-hydroxy-19-norpregn-4-en-3-one,
10-hydroxy-19-norpregn-4-en-20-yn-3-one,
10-hydroxy-19-norpregna-4,16,20-trien-3-one,
10-hydroxy-19-norpregna-4,17(20),20-trien-3-one,
10-hydroxy-19-norpregna-4,16-dien-20-yn-3-one,
3-methoxy-19-norpregna-2,5(10),16-triene,
3-methoxy-19-norpregna-2,5(10)-diene,
3-methoxy-19-norpregna-2,5(10)-dien-20-yne,
3-methoxy-19-norpregna-2,5(10),16,20-tetraene,
3-methoxy-19-norpregna-2,5(10),17(20),20-tetraene,
3-methoxy-19-norpregna-2,5(10),16-trien-20-yne,
19-norpregna-1,3,5(10),7,20-pentaen-3-ol,
19-norpregna-1,3,5(10),7,17(20)-pentaen-3-ol,
19-norpregna-1,3,5(10),7,16-pentaen-3-ol,
19-norpregna-1,3,5(10),7,-tetraen-3-ol,
19-norpregna-1,3,5(10),7,-tetraen-20-yn-3-ol,
19-norpregna-1,3,5(10),7,16,20-hexaen-3-ol,
19-norpregna-1,3,5(10),7,17(20),20-hexaen-3-ol,
19-norpregna-1,3,5(10),7,16-pentaen-20-yn-3-ol,
19-norpregna-1,3,5,7,9,20-hexaen-3-ol,
19-norpregna-1,3,5,7,9,17(20)-hexaen-3-ol,
19-norpregna-1,3,5,7,9,16-hexaen-3-ol,
19-norpregna-1,3,5,7,9-pentaen-3-ol,
19-norpregna-1,3,5,7,9-pentaen-20-yn-3-ol,
19-norpregna-1,3,5,7,9,16,20-heptaen-3-ol,
19-norpregna-1,3,5,7,9,17(20),20-heptaen-3-ol,
19-norpregna-1,3,5,7,9,16-hexaen-20-yn-3-ol,
19-norpregna-5(10),20-dien-3-ol,
19-norpregna-5(10),17(20)-dien-3-ol,
19-norpregna-5(10),16-dien-3-ol,
19-norpregn-5(10)-en-3-ol,
19-norpregna-5(10)-en-2-yn-3-ol,
19-norpregna-5(10),16,20-trien-3-ol,
19-norpregna-5(10),17(20),20-trien-3-ol,
19-norpregna-5(10),16-dien-20-yn-3-ol,
19-norpregna-1,3,5(10),20-tetraene-3,6-diol,
19-norpregna-1,3,5(10),17(20)-tetraene-3,6-diol,
19-norpregna-1,3,5(10),16-tetraene-3,6-diol,
19-norpregna-1,3,5(10)-triene-3,6-diol
19-norpregna-1,3,5(10)-trien-20-yne-3,6-diol,
19-norpregna-1,3,5(10),16,20-pentaene-3,6-diol,
19-norpregna-1,3,5(10),17(20),20 pentaene 3,6-diol,
19-norpregna-1,3,5(10),16-tetraen-20-yne-3,6-diol,
19-norpregna-5(10),20-dien-3-one,
19-norpregna-5(10),16-dien-3-one,
19-norpregn-5(10)-en-3-one,
19-norpregna-5(10)-16,20-trien-3-one,
19-norpregna-5(10),17(20),20-trien-3-one, and
19-norpregna-5(10),16-dien-20-yn-3-one.

### Examples

### Example 1

### Process for Making Pheromone-Containing Chocolate

First, cocoa beans are milled and ground to form a chocolate liquor, a liquefied form of pure chocolate flavor. The main ingredients in chocolate are the chocolate liquor, cocoa butter, sugar and milk. Chocolate liquor is a liquefied form of milled and grinded cocoa beans. These ingredients are mixed together and then dried into a coarse powder known as chocolate crumb. One or more pheromones may be added to this mixture in forming the chocolate crumb. Cocoa butter is then added to the powder to bring out the rich taste and creamy texture of the chocolate. The crumb then travels through special steel rollers which grind and refine the mixture, making it smoother. The crumb becomes a thick liquid called chocolate paste. The paste is poured into huge vats called conches. Once inside the conche, large granite rollers smooth out the gritty particles from the crumb. This process can take anywhere from 24 to 72 hours to complete. At this point, the chocolate paste has the smooth, familiar look of milk chocolate and it's ready to be molded into bars, bite-sized morsels, etc. The paste is then tempered, or cooled in a controlled manner to the right texture and consistency. Other ingredients, like almonds or peanuts, can be mixed into the paste during tempering or added directly to the molds.

### Example 2

### Process for Making Pheromone-Containing Lollipops

A marble slab or upside-down cookie sheet is prepared by covering it with parchment paper and spraying it with oil. If molds are being used, the molds are prepared with lollipop sticks, sprayed with oil, and place on a cookie sheet or marble slab. Over medium heat in a pan stir together sugar, corn syrup, water, cream of tarter (potassium bitartrate) until the sugar crystals dissolve. Continue to stir using a pastry brush dampened with warm water to dissolve any sugar crystals clinging to the sides of the pan and then stop stirring as soon as the syrup begins to boil. A candy thermometer is placed in the pan, being careful not to let it touch the bottom or sides, and the syrup is brought to a boil without stirring until the thermometer just reaches 300° F. (hard-crack stage). The pan is then removed from the heat immediately and the syrup is cooled to about 275° F. before adding flavor, color, and citric acid (adding it sooner causes most of the flavor to cook away). One or more pheromone compositions are also added at this time. The syrup is then quickly poured the prepared molds and left to cool for about 10 minutes. II molds are not being used, small (2-inch) circles are poured onto the prepared marble slab or cookie sheet and a lollipop stick is placed in each one, twisting the stick to be sure it's covered with candy. Allow the lollipops cool for at least 10 minutes, until they are hard. The lollipops should then be wrapped individually in plastic wrap or cellophane, sealed with tape or twist ties and the stored in a cool, dry place.

### Example 3

### Process for Making Pheromone-Containing Lipstick

First, raw ingredients for the lipstick are melted and mixed—separately because of the different types of ingredients used. One mixture contains the solvents, a second contains the oils, and a third contains the fats and waxy materials. These are heated in separate stainless steel or ceramic containers. The solvent solution and liquid oils are then mixed with the color pigments. The mixture passes through a roller mill, grinding the pigment to avoid a “grainy” feel to the lipstick. This process introduces air into the oil and pigment mixture, so mechanical working of the mixture is required. The mixture is stirred for several hours; at this point some producers use vacuum equipment to withdraw the air. After the pigment mass is ground and mixed, it is added to the hot wax mass until a uniform color and consistency is obtained. The fluid lipstick can then be strained and molded, or it may be poured into pans and stored for future molding. If the fluid lipstick is to be used immediately, the melt is maintained at temperature, with agitation, so that trapped air escapes. If the lipstick mass is stored, before it is used it must be reheated, checked for color consistency, and adjusted to specifications, then maintained at the melt temperature (with agitation) until it can be poured. Once the lipstick mass is mixed and free of air, it is ready to be poured into the tube. A variety of machine setups are used, depending on the equipment that the manufacturer has, but high volume batches are generally run through a melter that agitates the lipstick mass and maintains it as a liquid. For smaller, manually run batches, the mass is maintained at the desired mix temperature, with agitation, in a melter controlled by an operator. The melted mass is dispensed into a mold, which consists of the bottom portion of the metal or plastic tube and a shaping portion that fits snugly with the tube. Lipstick is poured “upside down” so that the bottom of the tube is at the top of the mold. Any excess is scraped from the mold. The lipstick is cooled (automated molds are kept cold; manually produced molds are transferred to a refrigeration unit) and separated from the mold, and the bottom of the tube is sealed. The lipstick then passes through a flaming cabinet (or is flamed by hand) to seal pinholes and improve the finish. The lipstick is visually inspected for air holes, mold separation lines, or blemishes, and is reworked if necessary. If necessary, the lipstick is reworked. Lipstick is reworked by hand with a spatula to remove air, blemishes, etc. This can be done in-line, or the tube can be removed from the manufacturing process and reworked. After the lipstick is retracted and the tube is capped, the lipstick is ready for labeling and packaging. Labels identify the batch and are applied as part of the automated operation.

The final step in the manufacturing process is the packaging of the lipstick tube. A variety of packaging options are available, ranging from bulk packs to individual packs, and including packaging as a component in a makeup kit or special promotional offering. Packaging for lipsticks varies, depending on the desired point of sale in the retail outlet. Packaging may or may not be highly automated, and the package used depends on the end use of the product rather than on the manufacturing process.

### Example 4

### Process for Making Body Wash Having a Human Pheromone Component

A typical body wash product according to an embodiment of the present invention comprises a formulation having the following ingredients and manufacturing procedure:

| Phase | Ingredient | Weight % |
|---|---|---|
| A | Water | q.s. |
| | Polyquaternium-10 | 0.40 |
| B | Sodium Laureth Sulfate (38% aqueous) | 40.00 |
| | Cocamidopropyl Betaine (30% aqueous) | 9.00 |
| | Glycerin | 2.00 |
| | Tetrasodium EDTA | 0.10 |
| | PEG-8 | 3.00 |
| | Citric Acid | 0.10 |
| | Sodium Chloride | 1.00 |
| C | Fragrance | 0.20 |
| | PPG-2 Hydroxyethyl Coco/Isostearamide | 0.50 |
| D | Cocamidopropyl PG-Dimonium Chloride | 0.50 |
| E | Androstadienone | q.s. |
| F | Polyquaternium-7 (9% aqueous) | 1.50 |
| G | DMDM Hydantoin (50% aqueous) | 1.00 |
| | | 100.00 |

Manufacturing Procedure:
1. Propeller mix Phase A water to form a vortex. Sprinkle the polyquaternium-10 into the vortex. When the polyquaternium-10 is completely wetted, decrease the propeller speed to just eliminate the vortex and mix until the polyquaternium-10 is completely dissolved.
2. Add Phase B into Phase A. Propeller mix Phase AB, without vortexing to 78-80° C. Hold mixing and temperature until all dissolved and clear.
3. Propeller mix and force cool Phase AB to 50-54° C.
4. Separately, mix Phase C until uniform. Add pre-mixed PhaseC into mixing Phase AB at 50-54° C.
5. Propeller mix and force cool Phase ABC to 22-24° C., adding Phases D, E, F and G separately and in that order beginning at 40° C.
6. Store product in tightly sealed containers.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all reasonable modifications.

1. A body wash having human pheromone component, wherein the body wash comprises:
a. a primary surfactant;
b. a secondary surfactant;
c. one or more specialty ingredients;
d. one or more additives and/or minor ingredients

2. The body wash as in claim 1, wherein the primary surfactant is an anionic surfactant.

3. The body was as in claim 2 wherein the anionic surfactant is selected from the group consisting of alkyl sulfates and alkyl ether sulfates.

4. The body wash as in claim 3, wherein the alkyl sulfates and alkyl ether sulfates further comprise sodium/ammonium lauryl sulfates and sodium/ammonium laureth sulfates.

5. The body wash as in claim 2, wherein the primary surfactant is sodium laureth sulfate.

6. The body wash as in claim 1, further comprising a nonionic surfactant.

7. The body wash as in claim 1, further comprising an amphoteric surfactant, a zwitterionic surfactant, or combinations thereof.

8. The body wash as in claim 1, wherein the secondary surfactant is selected from the group consisting of salts and betaines.

9. The body wash as in claim 8, wherein the secondary surfactant is selected from Cocamidopropyl betaine and PPG-2 hydroxyethyl coco/isostearamide.

10. The body wash as in claim 1, wherein the specialty ingredients include one or more of the following: conditioning agents, proteins, vitamins, moisturizers, humectants, botanicals and natural oils, coloring agents, pH adjusters and chelating agents.

11. The body wash as in claim 1, further comprising an emulsifying agent.

12. The body wash as in claim 11, wherein said emulsifying agent is propylene glycol cocoate (PEG-8).

13. The body wash as in claim 1, further comprising one or more amphoteric and/or zwitterionic surfactants.
